(19) United States
(12) Patent Application Publication
(10) Pub. No.: US 2009/0240238 A1
(43) Pub. Date: Sep. 24, 2009

(54) Clot Retrieval Mechanism

(75) Inventors: Todd Grodrian, Santa Rosa, CA (US); Joseph Berglund, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/054,170

(22) Filed: Mar. 24, 2008

(51) Int. Cl. A61M 1/00; A61B 17/24

(52) U.S. Cl. 604/540; 606/113

(57) ABSTRACT

A clot retrieval device for removing thrombi or other emboli from a body lumen is disclosed. The clot retrieval device includes an elongate shaft portion, a self-expanding snare disposed at a distal end of the shaft portion and a flexible bag portion attached to the self-expanding snare. The snare may be a loop or circlet formed in a distal end of the elongate shaft portion or may be a separate component attached thereto. An opening of the bag portion for receiving the thrombi or emboli there through faces proximally when the snare is fully deployed. The bag portion is made of a flexible, non-porous material, such as a balloon-type or elastomeric material, that is foldable or otherwise collapsible to achieve a low-profile while the clot retrieval device is being tracked to the treatment site through the body lumen.

### FIELD OF THE INVENTION

The present invention is directed to a clot retrieval and removal system for percutaneous removal of clots or other emboli from a body lumen.

### BACKGROUND OF THE INVENTION

Human blood vessels often become occluded (blocked) or stenotic (narrowed) by plaque, thrombi, deposits, or other substances, which reduce the blood carrying capacity of the vessel. For instance, a stenosis may be formed by an atheroma which is typically a harder, calcified substance which forms on the lumen walls of the blood vessel. Also, the stenosis can be formed of a thrombus material which is typically much softer than an atheroma, but can nonetheless cause restricted blood flow in the lumen of the blood vessel. Should the blockage occur at a critical place in the circulatory system, serious and permanent injury, or even death, can occur. To prevent this, some form of medical intervention is usually performed when significant occlusion is detected.

Percutaneous interventional procedures developed to treat occlusive vascular disease, such as angioplasty and stenting, often dislodge material from the vessel walls. This dislodged material, known as emboli, enters the bloodstream, and may be large enough to occlude smaller downstream vessels, potentially blocking blood flow to tissue. The resulting ischemia poses a serious threat to the health or life of a patient if the blockage occurs in critical tissue, such as the heart, lungs, or brain. In order to reduce the risk of embolization, guidewire filter devices or embolic protection devices that can be temporarily placed within the vascular system to collect and retrieve plaque, thrombus and other embolic particulate, which may be dislodged during angioplasty, stenting or other procedures, are known.

Another method of treating stenotic vasculature is removal of the stenosis by thrombectomy or atherectomy. During thrombectomy and atherectomy, the stenosis is mechanically cut or abraded away from the vessel. However, such procedures often require the use of some type of embolic protection device to capture emboli released from the treatment site.

In certain presentations, it may be appropriate or desirable to remove an emboli or thrombi in one operation, for example, removal of a cerebral thrombosis and/or other emboli blocking blood flow to the brain that are associated with ischemic stroke. In another example, removal of an emboli or thrombi in one operation may be desirable when the blockage occurs in a narrow or tortuous portion of the vasculature, which multiple more complex medical devices cannot easily access, or when the stenosis is of a softer thrombus composition. In addition, a medical device for removing thrombi or emboli from within a vascular system that eliminates the need for an additional filtering device would be desirable. As such embodiments of the present invention are directed to a medical device that provides a low profile self-expanding vascular device useful for thrombectomy and embolectomy that does not include a filtering element.

### BRIEF SUMMARY OF THE INVENTION

Embodiments of the present invention are directed to a clot retrieval device for removing thrombi or other emboli from a body lumen. The clot retrieval device includes an elongate shaft portion, a self-expanding snare disposed at a distal end of the shaft portion and a flexible bag portion attached to the self-expanding snare. The snare may be a loop or circlet formed in a distal end of the elongate shaft portion or may be a separate component attached thereto. Preferably at least the self-expanding snare is formed of a shape memory material. An opening of the bag portion for receiving the thrombi or emboli there through faces proximally when the snare is fully deployed. The bag portion is made of a flexible, non-porous material, such as a balloon-type or elastomeric material, that is foldable or otherwise collapsible to achieve a low-profile for tracking through the body lumen.

Embodiments of the present invention are directed to methods of using the clot retrieval device described in the previous paragraph. In an embodiment, a method of removing a clot or other emboli from a body lumen includes the steps of tracking a clot retrieval device having an elongate shaft portion, a self-expanding snare and a non-porous bag portion to a treatment site within the body lumen proximate the clot. The clot retrieval device is then deployed distal of the clot to allow the snare to reach an expanded configuration such that an opening of the bag portion faces proximally toward the clot. The snare is manipulated to envelope the clot within the bag portion of the clot retrieval device for subsequent removal from the body lumen. The method may include drawing the snare proximally over the thrombi or emboli and/or using a secondary device tracked proximal of the clot to push the thrombi or emboli through the snare into the bag portion of the device. In another embodiment, a balloon catheter may be used to restrict or prevent blood flow through the treatment site to aid in easing the snare over the clot or emboli. In addition, a balloon of the balloon catheter may be inflated within the body lumen to be in contact with the clot and act as a “stop” to prevent proximal migration of the clot during capture by the snare of the clot retrieval device. Such a method may help to prevent damage to a wall of the body lumen.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 illustrates a side view of a medical device according to an embodiment of the present invention.

FIG. 2 illustrates an expanded, partial cross-sectional view of a distal portion of the medical device of FIG. 1 tracked proximate a clot within a body lumen.

FIG. 2A depicts an end view of a clot retrieval device depicted in FIG. 2 in accordance with an embodiment of the present invention.

FIG. 3 illustrates a clot retrieval device according to an embodiment of the present invention partially deployed distal of the clot of FIG. 2.

FIG. 4 illustrates the clot retrieval device of FIG. 3 in a fully deployed configuration.

FIG. 5 illustrates the clot retrieval device of FIG. 3 enveloping the clot.

FIG. 6 illustrates a side view of a clot retrieval device in accordance with another embodiment of the present invention.

FIG. 6A depicts an end view of the snare of the clot retrieval device of FIG. 6 in an unexpanded configuration.

FIG. 7 is a flow chart depicting various methods in accordance with embodiments of the present invention.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. Furthermore, there is no intention to be bound by any expressed or implied theory presented in the preceding technical field, background, brief summary or the following detailed description.

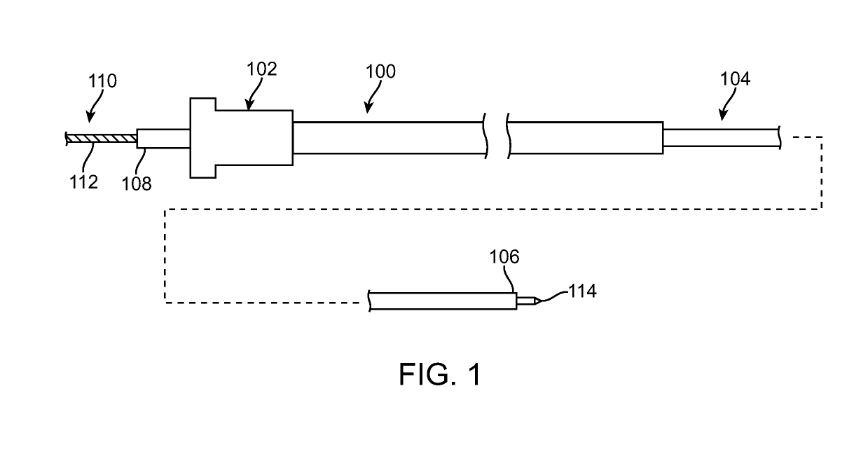

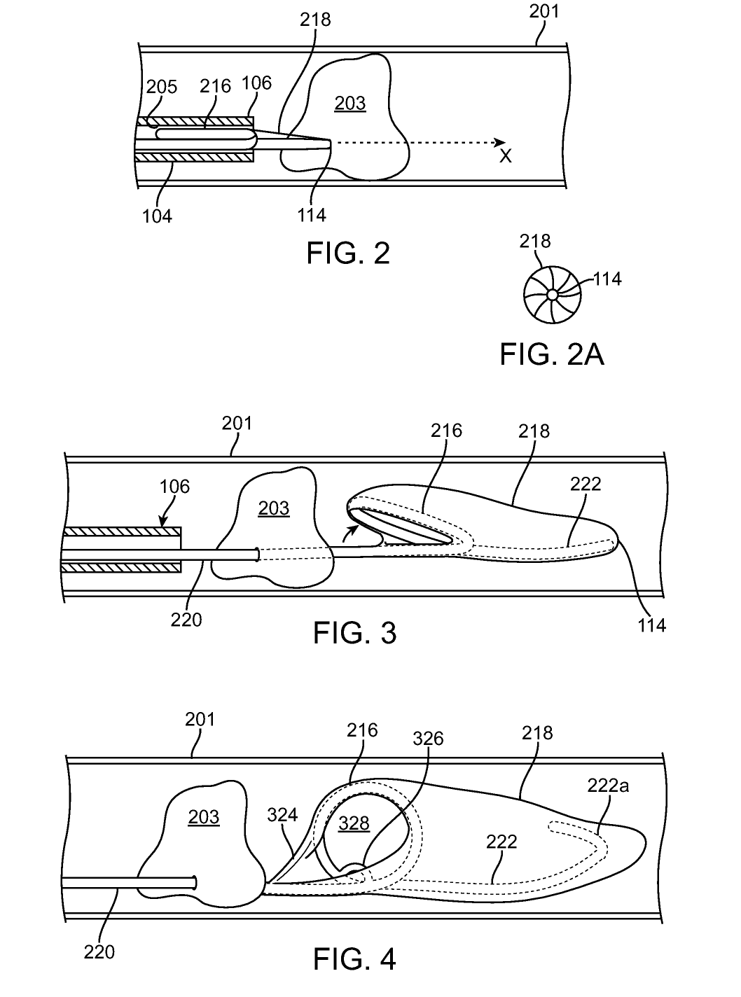

FIG. 1 illustrates a side view of a system for retrieving a clot or emboli from a body lumen in accordance with an embodiment of the present invention, with FIG. 2 illustrating an enlarged partial cross-sectional view of a distal portion of the system situated within a body vessel 201 proximate a clot or other emboli 203. The system includes a single-lumen guide catheter 100 having a hub 102 for attaching to a Tuohy-Borst adapter (not shown) at the site of entry into a body lumen, as would be known to one of ordinary skill in the art. Suitable guide catheters that may be used in embodiments of the present invention include those shown and described in U.S. Pat. No. 5,902,287 to Martin and U.S. Pat. No. 5,964,971 to Lunn, which are hereby incorporated by reference in their entirety. A single-lumen delivery or sheath catheter 104 is slidably received within the lumen of guide catheter 104 and has a proximal end 108 that remains proximal of hub 102 and a distal end 106 that is tracked through the vasculature to the point of treatment, i.e., the site of the thrombi or emboli 203 to be removed. Suitable delivery or sheath catheters that may be used in embodiments of the present invention include those shown and described in U.S. Patent Publ. No. 2007/0032815 to DeVaux and U.S. Patent Publ. No. 2005/0004553 to Douk, which are hereby incorporated by reference in their entirety.

A clot retrieval device 110 is shown slidably received within lumen 205 of sheath catheter 104 and includes a proximal end 112 that remains outside the patient proximate hub 102 and a distal end 114 that is positionable at the point of treatment proximal of clot 203. In FIG. 1, distal end 114 of clot retrieval device 110 is shown protruding slightly from distal end 106 of sheath catheter 104, but it should be understood that this is for illustrative purposes only and that during use while sheath catheter 104 is being advanced through the vasculature clot retrieval device 110 would be maintained within lumen 205 until deployment. In FIG. 2, distal end 114 of clot retrieval device 110 is shown being advanced distally from lumen 205 of sheath catheter 104 in the direction of arrow X, such that distal end 114 pierces and passes through clot 203 while clot retrieval device 110 is still in an unexpanded configuration.

In another embodiment, clot retrieval device 110 may utilize a restraining or delivery sheath to maintain the device in its collapsed configuration, in addition to or instead of sheath catheter 104, such that once a distal end of the restraining sheath is retracted by the clinician or the sheath is otherwise removed, the expandable or deployable portion of the device will transform into its fully expanded configuration, as will be explained in greater detail below with reference to FIGS. 3 and 4. In an embodiment, an RF energy source may be used to cut through the restraining sheath to allow expansion of the device.

With reference to FIGS. 3 and 4, clot retrieval device 110 includes an elongate shaft portion 220, a snare or wire circlet 216 and a balloon-type bag or pouch portion 218. Elongate shaft portion 220 extends from proximal end 112 to snare 216 and may be a solid wire, a hollow tube or of another construction suitable for a guidewire. Elongate shaft portion 220 must be flexible enough to navigate the tortuous pathways of the vascular system and yet have pushability. As such to impart improved pushability to clot retrieval device 110, elongate shaft portion 220 may be a solid core wire formed from a material such as stainless steel, a nickel-based super alloy, a spring steel alloy, particularly a composition sold under the trademark ELIGILOY, titanium, MP35N or nitinol. In addition, a core wire that forms elongate shaft portion 220 may be ground down and have several diameters in its profile in order to provide a transition in stiffness along its length.

In another embodiment, at least a proximal portion of elongate shaft portion 220 may be of a thin-walled, tubular structure of a metallic material, such as stainless steel, a spring steel alloy, particularly a composition sold under the trademark ELIGILOY, MP35N, titanium or nitinol. In an embodiment, a metallic tube commonly referred to as hypodermic tubing or a hypotube may be used. In an alternate embodiment, elongate shaft portion 220 may include a proximal tubular structure made from a polymeric material, such as polyethylene block amide copolymer, polyvinyl chloride, polyethylene, polyethylene terephthalate, polyamide, polyimide, polypropylene, polytetrafluoroethylene, polycarbonates, and polyurethanes, alone or in combination with a metal reinforced polymer shaft portion utilizing one or more of the aforementioned materials.

Snare 216 may be a loop formed in elongate shaft portion 220 proximate the distal end 114 of the clot retrieval device 110. The transformation of snare 216 from a compressed, delivery configuration to an expanded, deployed configuration may be impelled by external mechanical means, such as in the manner of a lasso wherein the clinician distally feeds an additional length of shaft portion 220 until snare 216 achieves an expanded configuration, or by self-shaping memory to be self-expanding. Preferably, snare or circlet 216 is self-expanding, meaning that snare or circlet 216 has a mechanical memory to return to the expanded, or deployed configuration. Mechanical memory may be imparted to the metal of snare 216 by thermal treatment to achieve a spring temper in stainless steel, for example, or to set a shape memory in a susceptible metal alloy, such as nitinol.

In an alternate embodiment, the snare may be a separate component that is attached to a distal end of the elongate shaft portion. In such an embodiment, the snare may be formed from a different material than the remainder of the clot retrieval device. In certain instances, a separately formed snare may achieve manufacturing and/or performance advantages over an integral snare and shaft component.

Balloon-type bag or pouch portion 218 of clot retrieval device 110 is of a balloon or other flexible material that is nonporous and is formed in a conical or windsock-like shape. In accordance with embodiments of clot retrieval devices described herein, a nonporous material is a continuous material, such as a fabric or film, with a pore structure that is small enough so as to restrict transport of thrombi, leukocytes, erythrocytes, and similar solid blood constituents therethrough. In other words, the nonporous material has no openings or holes formed therein that would allow blood flow through the material. In an embodiment, bag portion 218 may be of a flexible, non-elastic or elastomeric material to include polymeric materials used in angioplasty balloons or covered grafts, such as polyamides, to include certain nylons, polyurethanes, polytetrafluoroethylene, polyesters, including Dacron, and polyimides. With reference to FIG. 2A, bag portion 218 may be formed to fold around snare 216 in a predetermined manner, such as the spiral “star” shape shown, to achieve a low-profile and ease of deployment. In order to achieve a symmetrically consistent folded profile, bag portion 218 may be formed of material and in a manner as shown and described with respect to angioplasty balloons in U.S. Pat. No. 5,350,361 to Tsukashima et al., which is incorporated by reference herein in its entirety.

With reference to FIGS. 3 and 4, clot retrieval device 110 is maneuvered to be distal of clot 203 such that the device achieves an expanded configuration distal of the clot or emboli. Bag portion 218 is shown attached to snare 216 such that an opening 328 of bag portion 218 faces proximally toward clot 203 when the snare 216 is fully deployed. In order to prevent over enlargement of snare 216, a ring or circular clip 326 closely encircles the loop or winding that forms snare 216 to aid in defining or fixing a diameter of opening 328. Ring 326 is secured to shaft portion 220. In an embodiment, ring 326 may be sized to allow some expansion of the loop that forms snare 216 thereby allowing snare 216 to expand to conform to blood vessels of varying diameters. In another embodiment, ring 326 may restrict movement of the loop that forms snare 216 so that snare 216 may be preset in size such that when fully expanded snare 216 may not come into contact with the walls of vessel 201. Alternatively, a tether or wire (not shown) may be attached to ring 326 to permit a clinician to manipulate the diameter of the loop to open or close snare 216.

In an embodiment where snare 216 is of nitinol, bag portion 218 may be attached to snare 216 either by coating snare 216 with a biocompatible polymer or by surrounding snare 216 with a suitable polymeric sleeve such that a material of bag portion 218 may be thermally bonded thereto. In addition, webs 324 of a balloon or other flexible material may be attached between a proximal end of snare 216 and elongate shaft portion 220 to add structural support to snare 216 and aid in maintaining proper orientation of ring 326 and proximal opening 328 during capture of the clot.

A small flexible end wire or tail portion 222 may distally extend within bag portion 218, from snare 216 to distal end 114 of clot retrieval device 110, in order to maintain or define a length of bag portion 218 in its expanded configuration. In an alternate embodiment, as shown by the dotted line in FIG. 4, tail portion 222 may include a curved portion 222a that aids in defining an interior space of bag portion 218.

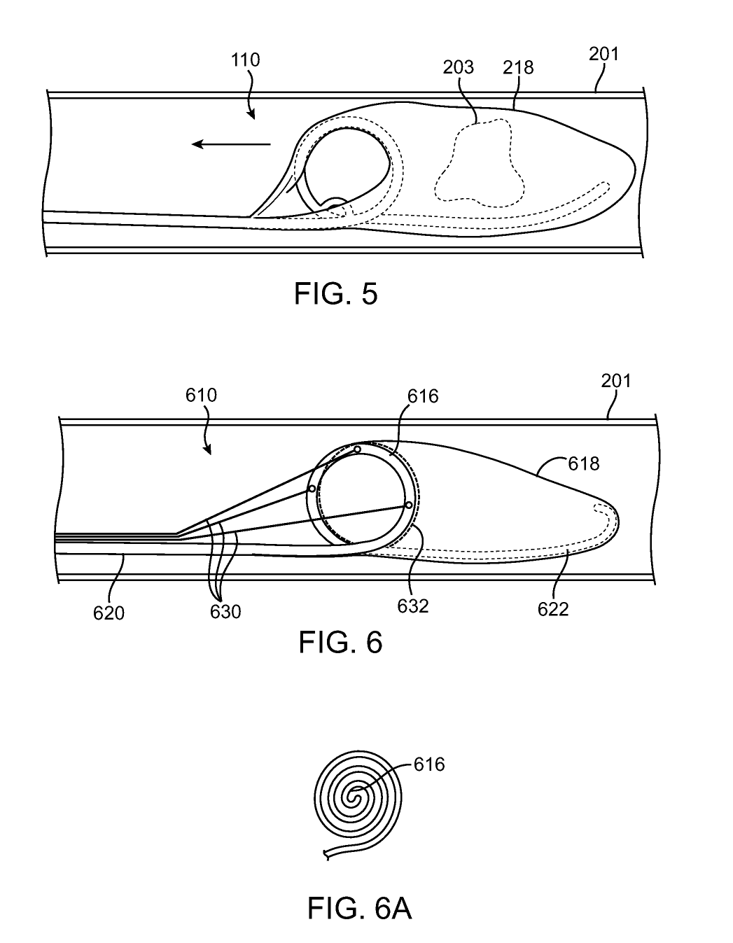

In FIG. 5, clot retrieval device 110 is shown pulled proximally over clot 203 in vessel 210 such that clot 203 is enveloped within bag portion 218. It should be understood that bag portion 218 is not necessarily of a transparent material and that clot 203 is viewable therethrough in FIG. 5 for illustrative purposes only.

FIG. 6 illustrates a clot retrieval device 610 in accordance with another embodiment of the present invention, with FIG. 6A depicting an end view of clot retrieval device 610 in a spirally coiled, unexpanded configuration. Clot retrieval device 610 includes elongate shaft portion 620, snare 616 and non-porous, balloon-type bag portion 618, each of which may be made of the various materials previously described with reference to the embodiment of FIGS. 2-5. Elongate shaft portion 620 may be constructed as in the previous embodiment with a winding or loop forming snare 616 and tail portion 622. However, as shown in FIG. 6A, snare 616 is wound upon itself or spirally coiled to achieve the compressed or unexpanded configuration of clot retrieval device 610. As such, snare 616 unwinds to the diameter of vessel 201 to achieve the expanded configuration of clot retrieval device 610, as shown in FIG. 6. In this manner snare 616 is able to fit variable diameters of vasculature. Bag portion 618 may be bonded, such as by thermal bonding, to snare 616 in a manner as previously described such that bag portion 618 may be tightly coiled along with snare 618 to achieve a low-profile and for ease of deployment.

Tethers 630 are attached at each of their distal ends to snare 616 and have proximal ends (not shown) that extend out of the patient for manipulation and control by the clinician. In other words, tethers 630 proximally extending adjacent elongate shaft portion 620 for substantially the length of clot retrieval device 610. In an embodiment where a proximal portion of elongate shaft portion 620 is a hollow tube, a distal port may be provided to allow a proximal length of tethers 630 to enter a lumen of elongate shaft portion 620 to be accessible to a clinician via a proximal port therein. Tethers 630 facilitate manipulation, clot retrieval, and extraction of clot retrieval device 610 by allowing a clinician additional control of the device. Tethers 630 may be thin metallic wires or strands of material. Although three tethers 630 are shown, it should be understood that fewer or more tethers may be utilized without departing from the scope of this embodiment of the present invention.

In another embodiment, in order to accommodate the unwinding of snare 616, bag portion 618 may be slidably attached thereto via hem 632. Hem 632 is sized and configured to allow the loop that forms snare 616 to enter and slidably extend through hem 632 unobstructed so that tail portion 622 sits within bag portion 618. Tethers 630 may be attached to hem 632, rather than snare 616, in order to allow snare 616 to freely slide/uncoil within hem 632. In an alternate embodiment wherein a tail portion is not utilized, hem 632 may be sized to accommodate unwinding/expansion of coiled snare 616 therein.

A method of using a clot retrieval device in accordance with an embodiment of the present invention is described as follows, with reference to FIGS. 3-5. A guidewire (not shown) is advanced into the patient's vasculature until the intended treatment site, i.e., the clot or emboli 203, is reached. A sheath or delivery catheter 104 is loaded with a clot retrieval device 110 in accordance with an embodiment of the present invention. The sheath catheter 104 is then back-loaded onto the guidewire and tracked thereover until a distal end 106 of the sheath catheter 104 is proximal of the clot or emboli 203 to be removed. The clot retrieval device 110 is then pushed distally from the sheath catheter lumen 205 to a position distal of the clot where the snare 216 of the device self-expands into its deployed configuration, as shown in FIGS. 3 and 4. With the snare 216 and bag portion 218 fully deployed within the vessel, the clot retrieval device 110 is manipulated, e.g., proximally retracted, such that the clot 203 is enveloped within the bag portion 218 of the device for removal.

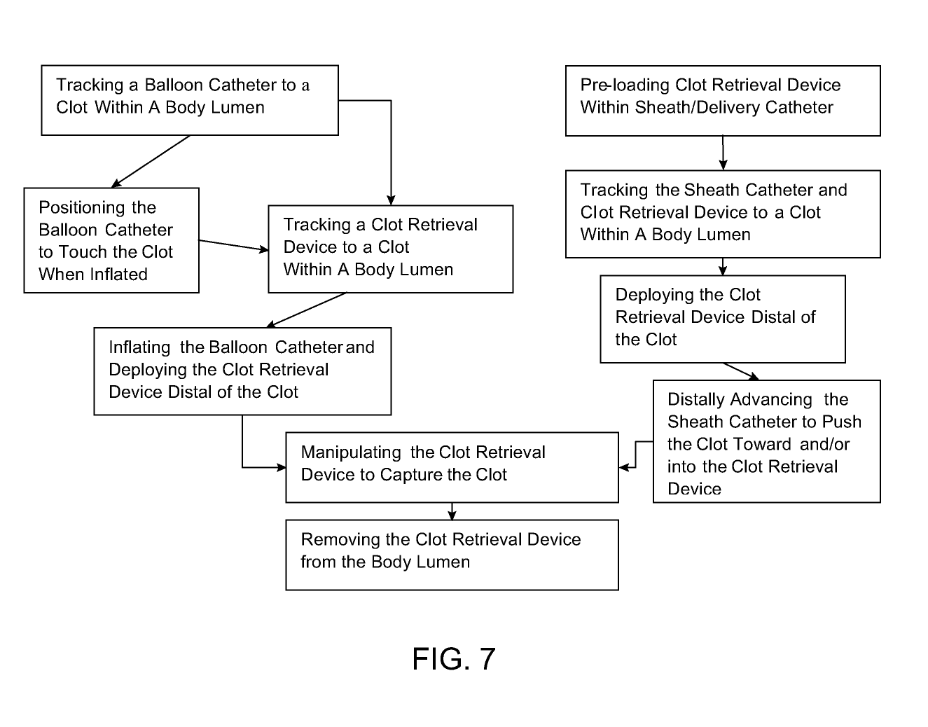

Alternatively, as represented in the method depicted on the right side of the flow chart of FIG. 7, after the clot retrieval device 110 has been deployed the sheath catheter 104 may be advanced distally to push the clot 203 through the snare 216 into the bag portion 218 of the device for removal. In either manner, the clot retrieval device 110 with the clot therein may then be retracted back into the sheath catheter 104 for subsequent removal from the vasculature. It would be understood by one of ordinary skill in the art that the sheath catheter 104 may be removed prior to the manipulation of the clot retrieval device 110 over the clot, such that the clot retrieval device 110 with the clot therein is removed separately from the vasculature upon capture of the clot.

In a method according to another embodiment of the present invention, as represented in the method depicted on the left side of the flow chart of FIG. 7, the sheath catheter 104 may be removed from the vasculature after deployment of the clot retrieval device 110 and a balloon catheter (not shown) may be advanced over the guidewire, if still indwelling, or over the elongate shaft portion 220 of the clot retrieval device 110 until a distal end thereof is positioned in apposition with a proximal portion of the clot. One of ordinary skill in the art will recognize that any of a number of balloon catheters known in the art would be suitable for practicing this embodiment of the present invention. The balloon of the balloon catheter may then be inflated to a sufficient diameter to hold its longitudinal position within the vessel. The balloon may then act as a stop to prevent the clot from moving proximally away from the snare 216 and bag portion 218 of the clot retrieval device 110 to thereby ease the capture of the clot by the clot retrieval device 110. Additional advantages of this method are that the balloon stops or dramatically restricts blood flow thereby preventing the blood from carrying distally small portions of thrombi or emboli, which may break-off during positioning of the clot retrieval device; it also quells a “water-hammering” effect, which may tend to break-up the clot; and in certain areas of the vasculature may effectively create a backflow helping to dislodge the clot and easing removal thereof.

In a method according to another embodiment of the present invention, as represented in the alternate method depicted on the left side of the flow chart of FIG. 7, a large lumen balloon catheter (not shown), rather than a simple sheath catheter, may be initially tracked over the guidewire to be positioned proximal of the clot. A clot retrieval device 110 in accordance with an embodiment of the present invention may then be slidably advanced through the balloon catheter lumen to penetrate and pass through, or alternatively pass by, the clot such that the snare 216 and bag portion 218 of the clot retrieval device 110 expands distal of the clot. A balloon of the balloon catheter is then inflated to substantially decrease or entirely stop blood flow as the clot retrieval device 110 is proximally advanced to envelope the clot within the bag portion 218 of the device for removal from the vasculature. A large lumen balloon catheter suitable for practicing this method of the present invention is shown and described in U.S. Pat. No. 6,702,782 to Miller et al., which is hereby incorporated by reference herein in its entirety.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A clot retrieval device for removing thrombi from a body lumen, the device comprising:
an elongate shaft portion;
a self-expanding snare disposed at a distal end of the shaft portion; and
a flexible bag portion attached to the self-expanding snare such that an opening of the bag portion for receiving thrombi there through faces proximally when the snare is fully deployed, wherein the bag portion is made of a non-porous material.

2. The clot retrieval device of claim 1, wherein at least a distal segment of the shaft portion is a metallic wire and the snare is a loop or winding in the metallic wire.

3. The clot retrieval device of claim 2, wherein the metallic wire is of a super elastic material.

4. The clot retrieval device of claim 2, wherein the metallic wire is of a material selected from the group consisting of stainless steel, spring steel, MP35N, nitinol and titanium.

5. The clot retrieval device of claim 2, wherein the loop that forms the snare extends through a ring attached to the distal segment of the shaft portion.

6. The clot retrieval device of claim 2, wherein the snare may be wound into a spiral coil to achieve a low-profile configuration for delivery within the body lumen.

7. The clot retrieval device of claim 6, wherein the snare is slidably disposed within a hem in the material of the bag portion.

8. The clot retrieval device of claim 1, wherein the bag portion is of a flexible, non-elastic balloon-type material.

9. The clot retrieval device of claim 1, wherein the bag portion is of an elastomeric material.

10. The clot retrieval device of claim 1, wherein an end wire distally extends within the bag portion from the snare such that the end wire maintains a length of the bag portion when the snare is fully deployed.

11. The clot retrieval device of claim 10, wherein the bag portion has a conical shape.

12. The clot retrieval device of claim 1, further comprising:
a tether having a distal end attached to the snare with a length that proximally extends to a proximal end of the elongate shaft portion, wherein the tether may be controlled by a clinician to aid in retrieval of the thrombi.

13. The clot retrieval device of claim 12, wherein the tether is a thin wire.

14. The clot retrieval device of claim 1, wherein webs of a flexible material connect a proximal end of the snare to the elongate shaft portion.

15. A method of removing a clot from a body lumen comprising the steps of:
tracking a clot retrieval device having an elongate shaft portion, a self-expanding snare and a non-porous bag portion to a treatment site proximate the clot;
deploying the clot retrieval device distal of the clot to allow the snare to reach an expanded configuration such that an opening of the bag portion faces proximally toward the clot; and
manipulating the snare to envelope the clot within the bag portion of the clot retrieval device.

16. The method of claim 15, wherein the method further comprises the step of:
loading the clot retrieval device within a lumen of a sheath or delivery catheter to maintain the clot retrieval device in a delivery configuration such that the step of tracking includes tracking the sheath or delivery catheter with the clot retrieval device therein to the treatment site.

17. The method of claim 16, wherein the step of manipulating the snare includes distally advancing the sheath or delivery catheter to push the clot through the snare.

18. The method of claim 15, wherein the method further comprises the step of:
tracking a balloon catheter to the treatment site; and
inflating a balloon of the balloon catheter to impede or prevent blood flow at the treatment site, wherein the step of manipulating the snare includes distally retracting the snare over the clot while the blood flow is impeded or prevented.

19. The method of claim 18, wherein the step of tracking the balloon catheter includes positioning the balloon such that when the balloon is inflated a distal end of the balloon is in apposition with a proximal portion of the clot to prevent the clot from longitudinal movement as the snare is manipulated thereover.

20. The method of claim 15, wherein the clot retrieval device includes at least one proximally extending tether attached to the snare such that the step of manipulating the snare includes using the tether to control the snare.
